(19) United States
(12) Patent Application Publication
(10) Pub. No.: US 2007/0016292 A1
(43) Pub. Date: Jan. 18, 2007

(54) Epithelium treatment methods and devices for treating the epithelium

(76) Inventor: Edward Perez, Palo Alto, CA (US)

Correspondence Address: E. THOMAS WHEELOCK, P.O. BOX 61168, PALO ALTO, CA, 94306, US

(21) Appl. No.: 11/434,725

(22) Filed: May 15, 2006

Related U.S. Application Data
(63) Continuation-in-part of application No. PCT/US04/38186, filed on Nov. 15, 2004.
(63) Continuation-in-part of application No. PCT/US04/31231, filed on Sep. 22, 2004.
(60) Provisional application No. 60/519,903, filed on Nov. 14, 2003.

(51) Int. Cl. A61F 2/14; A61F 9/013; G02C 7/04

(52) U.S. Cl. 623/5.13; 606/166; 623/5.14; 351/160 R

(57) ABSTRACT

In general, the devices and methods described herein are useful in the field of ophthalmology. More particularly, the described methods and devices are useful in the field of refractive eye procedures, such as may be practiced when lifting or separating a portion of the epithelial layer or forming a pocket in the epithelial layer of the when introducing a contact lens beneath the epithelium or in conjunction with a corrective ocular laser treatment.

### RELATED APPLICATIONS

This is a continuation-in-part of PCT/US2004/038186, filed 15 Nov. 2004, and has priority from U.S. Provisional Application No. 60/519,903, filed 14 Nov. 2003, the entirety of which are incorporated by reference.

### FIELD

In general, the devices and methods described herein are useful in the field of ophthalmology. More particularly, the described methods and devices are useful in the field of refractive eye procedures, such as may be practiced when lifting or separating a portion of the epithelial layer or forming a pocket in the epithelial layer of the when introducing a contact lens beneath the epithelium or in conjunction with a corrective ocular laser treatment.

### BACKGROUND

The cornea is the outermost layer of the eye. It is a clear layer, which helps in focusing light to create images on the retina. Unlike many other body tissues, the cornea contains no blood vessels to nourish it or to protect it from infection. Instead, the cornea is comprised of cells and proteins, and receives its nourishment from tears and the aqueous humor that fills the chamber behind it. The cornea is comprised of five basic layers: the epithelium, the Bowman's layer, the stroma, the Descemet's membrane, and the endothelium. Each layer is thought to provide a separate and unique function.

The epithelium is the outermost layer of the cornea. It comprises about 10 percent of the cornea's tissue thickness and has two primary functions. First, the epithelium functions to block the passage of foreign materials into the eye. Second, the epithelium functions to provide a smooth surface, which absorbs oxygen and nutrients. The epithelium is filled with thousands of tiny nerve endings, which make the cornea extremely sensitive to pain when rubbed or scratched. The part of the epithelium that serves as the foundation on which the epithelial cells anchor and organize themselves is called the basement membrane.

Lying directly below the basement membrane of the epithelium is a transparent sheet of tissue known as Bowman's layer. The Bowman's layer is composed of strong layered protein fibers called collagen. Beneath the Bowman's layer is the stroma, which comprises about 90 percent of the cornea's thickness. It consists primarily of water and collagen (collagen I and

The collagen gives the cornea its strength, elasticity, and form. In addition, the shape, arrangement, and spacing of the collagen are important in producing the cornea's light-conducting transparency.

Under the stroma is the Descemet's membrane. The Descemet's membrane is a thin, but strong sheet of tissue that serves as a protective barrier against infection and injuries. The Descemet's membrane is composed of collagen fibers, which are of a different nature than those of the stroma, and is made by the cells that lie below it.

The endothelium is the innermost layer of the cornea. The thin layer of endothelial cells is important in keeping the cornea clear. The primary task of the endothelium is to pump excess fluid out of the stroma. Without this pumping action, the stroma would swell with water, become hazy, and ultimately opaque. In a healthy eye, a perfect balance is maintained between the fluid moving into the cornea and the fluid being pumped out of the cornea. Once endothelium cells are destroyed by disease or trauma, they are lost forever.

Usually the shape of the cornea and the eye are not perfect and the image on the retina is blurred or distorted. These imperfections are called refractive errors. There are three primary types of refractive errors: myopia (nearsightedness), hyperopia (farsightedness), and astigmatism (distortion of the image on the retina caused by corneal or lens irregularities).

Combinations of these refractive errors are common in many people. Glasses and contact lenses are designed to compensate for, and to temporarily correct, these errors. However, surgical procedures, such as LASIK, RK, PRK, and LASEK are also available.

LASIK stands for Laser-Assisted Keratomileusis. It is a procedure that permanently changes the shape of the cornea. During LASIK, a knife called a microkeratome is used to cut a flap in the cornea. A hinge is left at one end of this flap, which is folded back to reveal the stroma. An excimer laser is used to shape, or ablate, a portion of the stroma, and the flap is then replaced. The proper shaping of the stroma is dependent upon the type of refractive error the patient suffers from.

Radial Keratototomy (“RK”) and Photorefractive Keratectomy (“PRK”) are other refractive procedures used to reshape the cornea. In RK, a knife is used to cut tiny slits in the cornea, causing it to change its shape. PRK is similar to RK, except a laser is used to reshape the cornea. Often the same type of laser is used in LASIK and PRK procedures. The major difference between the two procedures is the way in which the stroma is exposed before it is ablated with a laser. In PRK, the epithelium is scraped away to expose the stromal layer underneath. In LASIK, a flap is cut in the stromal layer and the flap is folded back. RK and PRK are no longer common procedures.

LASEK stands for Laser Assisted Sub-Epithelial Keratectomy. With LASEK, no microkeratome is used, and no cut is made with a blade in the middle of the stroma. Essentially, LASEK may be thought of as a blend of the desirable features of the LASIK and PRK procedures. In LASEK, a dilute solution of alcohol is applied to loosen and remove the outermost surface of the epithelium. Once the epithelial layer has been removed, an excimer laser is then used to reshape the cornea, as in both LASIK and PRK. Upon completion of the excimer laser treatment, the epithelial layer is then returned to its original position.

In one of my previous applications, I described other methods for forming an epithelial flap, or removing an epithelial layer as a step of a refractive procedure, which are in some respects, superior to those methods described just above. That is, my methods typically involve the production of a pure epithelial flap. The plane of “separation” is just beneath the inferior cell membrane of the basal epithelial cell, and just above the Collagen I and Collagen III of the anterior corneal stroma. I refer to my methods of making a pure epithelial flap, or pocket, as epithelial delamination. These methods are described in application Ser. No. PCT/US03/01549 entitled, “Methods for Producing Epithelial Flaps on the Cornea and for Placement of Ocular Devices and Lenses Beneath and Epithelial Flap or Membrane, Epithelial Delaminating Devices, and Structures of Epithelium and Ocular Devices and Lenses,” which was filed on January 2003, and is hereby incorporated by reference in its entirety.

Epithelial delamination, as I have previously described may be performed by chemical, thermal, or mechanical devices and procedures. For example, osmotic blistering (e. g., with a 1 M solution) achieves a separation at the basal lamina (i. e., the lamina lucida) that results in the production of a pure epithelial flap. So does suction blistering. In addition, since the lamina lucida is the weakest link of adherence, mechanical force along the basement membrane results in a blunt dissection along the lamina lucida. Forceful introduction of a mechanical probe or fluid can be used to achieve a blunt dissection to create an epithelial flap.

### BRIEF DESCRIPTION OF THE DRAWINGS

FIG. 1A is a schematic of the eyeball.

FIG. 1B is an exploded view of the corneal layers.

FIG. 2A provides an illustration of an intact epithelial layer.

FIG. 2B provides an illustration of an epithelial layer that has been delaminated.

FIGS. 3A and 3B illustrate suitable devices for delaminating an epithelial layer.

FIG. 4A provides an illustration of laser ablation of the cornea after the epithelial layer has been delaminated.

FIG. 4B depicts an epithelial layer that has melted away in the area of corneal ablation.

FIGS. 5B, and 5D show bandage contact lenses suitable for use with the methods described herein.

FIGS. 6A and 6B depict various treatment methods in which the cells of the intact epithelial layer are used to help regenerate additional epithelial cells over the area of corneal ablation.

FIGS. 7A, 7B, and 7C show, respectively, a perspective view, a side cross-sectional view (across the vacuum ring), and a cross section view of the handle of a variation of my cooling device.

FIGS. 8A and 8B show, respectively, a side cross-sectional (across the vacuum ring), and a cross section view of the handle of another variation of my cooling device.

FIGS. 9, 10, and 11 show a side cross-sectional views (across the vacuum ring) of several variations of my cooling device.

FIG. 12 shows a side cross-sectional view of a variation of my cooling device included in a blunt dissector de-epithelization system.

FIGS. 13A and 13B show a side cross-sectional view and a perspective view of a variation of my cooling device included in a blunt dissector de-epithelization system suitable for forming an epithelial pocket. FIG. 13C shows such an epithelial pocket.

FIG. 14 shows a side cross-sectional view of a variation of my cooling device utilizing a thermoelectric cooling device such as a Peltier device.

### DETAILED DESCRIPTION

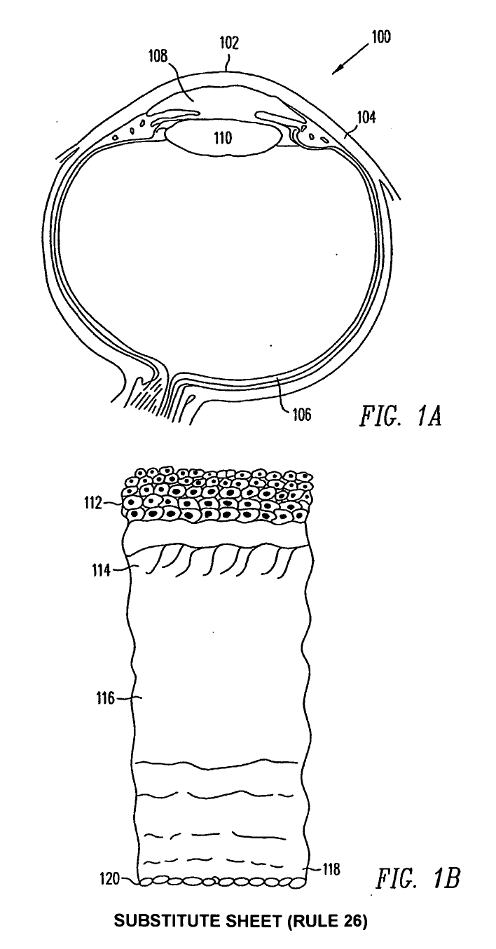

The eye is designed to focus light onto specialized receptors in the retina that turn quanta of light energy into nerve action potentials. As shown in FIG. 1A, the outermost layer of the eye is the cornea. The margins of the cornea merge with a tough fibrocollagenous sclera (104), referred to as the corneo-scleral layer. The cornea (102) is the portion of the corneo-scleral layer enclosing the anterior one-sixth of the eye. The smooth curvature of the cornea is the major focusing power of images on the retina (106) and this curvature provides much of the eye's 60 diopters of converging power.

As noted above, the cornea is an avascular structure and is sustained, in large part, by diffusion of nutrients and oxygen from the aqueous humor (108). Also shown in FIG. 1A is the lens (110). Shown in an exploded fashion by FIG. 1B are the five basic layers of the cornea: the epithelium (112), the Bowman's layer (114), the stroma (116), the Descemet's membrane (118), and the endothelium (120).

As previously described, many of the refractive eye procedures require that a portion of the epithelial layer be removed, or pushed aside, in order to access the underlying stroma for ablation. I have found that a preferred method of epithelial flap production involves the production of a pure epithelial flap or epithelial pocket, where the plane of “separation” is just beneath the inferior cell membrane of the basal epithelial cell, and just above the Collagen I and Collagen III of the anterior corneal stroma. I refer to my methods of making a pure epithelial flap or pocket, as epithelial delamination.

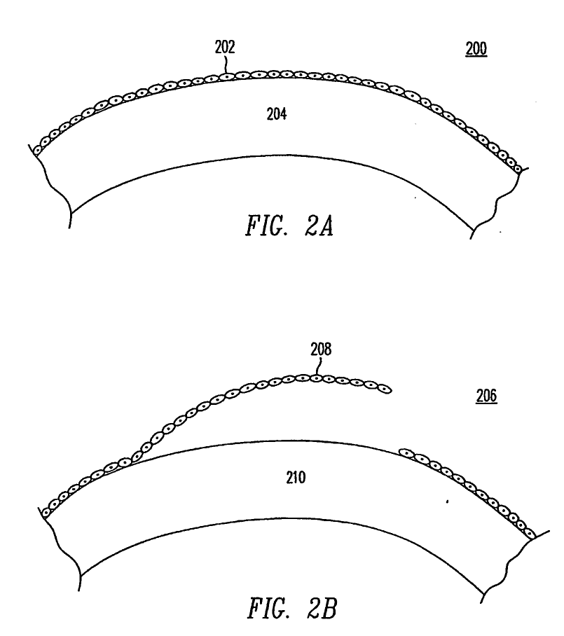

Shown in FIG. 2A, is a cornea (200) with its outermost epithelial layer (202) intact. In order to access the stroma (204) for ablation, the epithelial layer (202) may be partly removed, or pushed aside. FIG. 2B shows a cornea (206) after epithelial delamination. As shown there, a pure epithelial flap (208) has been produced, leaving stromal area (210) accessible for ablation or placement of a contact lens. Formation of an epithelial pocket (a subset of epithelial flap) may also easily be produced by, for instance, using a blunt delaminating device resembling a spatula, perhaps oscillating.

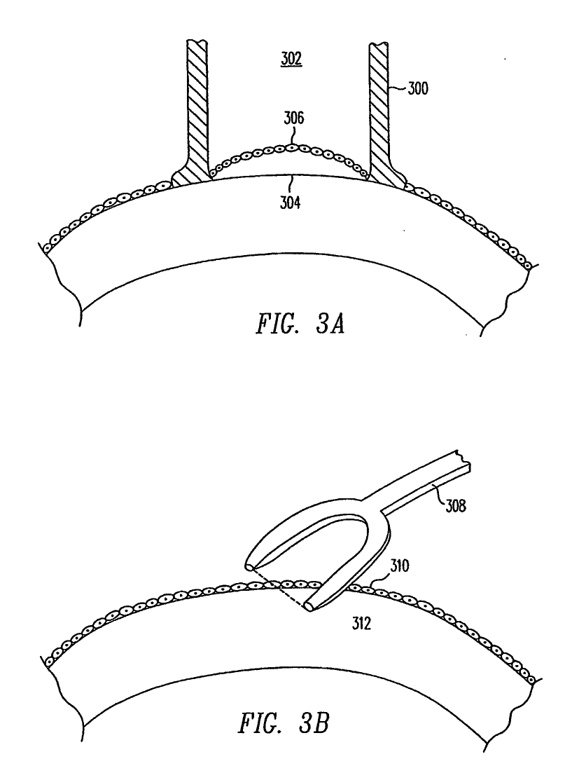

Epithelial delamination, as I have previously described may be performed by a variety of suitable techniques. For example, chemical, thermal, or mechanical devices and procedures may be used to delaminate the epithelium. Examples of suitable epithelial delamination techniques are shown in FIGS. 3A and 3B. Shown in FIG. 3A is a suction apparatus (300) for epithelial delamination.

The suction apparatus (300) includes a suction chamber that has an epithelial contact surface (304) and a vacuum source (not shown). In operation, the suction apparatus (300) is placed on the epithelial layer and the vacuum source is turned on. This results in the formation of a suction blister (306), and consequent epithelial flap.

Another suitable method of epithelial delamination is shown in FIG. 3B. Shown in FIG. 3B, is a blunt dissector (308). Blunt dissectors have non-cutting surfaces that are appropriate for placement between the epithelium and the collagenous stromal tissue (312). As used herein, the term “non-cutting” means that the blunt dissector does not have the ability to incise into the stroma of the cornea when used with normal force. I believe that my blunt dissectors separate the epithelium from the stromal layers of the cornea in the basal membrane zone at the natural point of weakest attachment, i.e., the lamina lucida.

Epithelial delamination may also be chemical in nature. I have found that suitable chemical compositions for epithelial delamination include vesicants such as 1 M hypertonic saline, ethanol, cantharidin, and CEES. Diluents may also be added to the composition prior to eye application. A suitable diluent for cantharidin is acetone. A suitable diluent for CEES is water or humidified air. Typically, as with cantharidin and CEES, the compounds work by destroying the basal epithelial cells themselves, but do not harm the epithelial cells that reside above the basal epithelial layer. If 1 M hypertonic saline is used, the basement membrane complex dissociates along the lamina lucida. Basal epithelial cells are generally not destroyed. Incubation of any epithelial cells in 1 M hypertonic saline achieves a pure separation of epithelium from the underlying connective tissue.

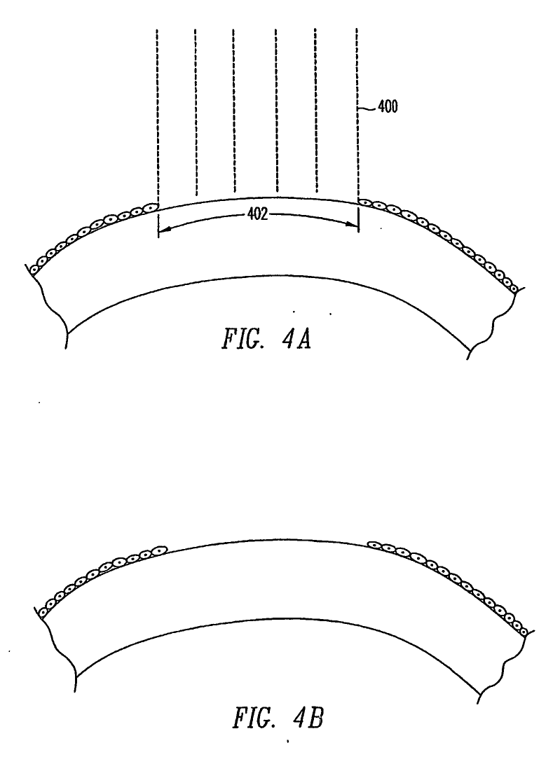

After a pure epithelial flap, or pocket, has been produced by any suitable delaminating technique, the stroma may then be shaped or ablated, or otherwise treated by a laser (400), as shown in FIG. 4A. The epithelial flap is then replaced and the eye allowed to heal. Alternatively, a sub-epithelial contact lens, perhaps a corrective lens, may be placed on the cornea and the epithelial flap replaced onto the center surface of the lens. In some instances, after laser procedures, a bandage contact lens (not shown here) is provided to aid in the healing process. However, we have sometimes, noted that after such laser-based refractive procedures, the epithelium of some patients undergoes. a “melting” in the center of the replaced flap. It may be the case that the epithelial cells in the “melted area” of the epithelial flap die. Often, this phenomenon is seen in the region right above the area in which the stroma has been ablated (402). As shown in FIG. 4B, the epithelial layer degrades leaving the cornea unprotected.

The methods described here are for treatment to the eye, cornea (de-epithelialized or not), or to the epithelial flap, to lessen, minimize, or prevent such epithelial degradation. Epithelial degradation may have been caused by a number of reasons. For example, the ablation, or delamination procedures could have disrupted or altered the natural cell biology of the Bowman's membrane. For example, these procedures may have destroyed certain adhesion molecules, which are necessary to ensure proper epithelial wound healing. In these instances, it may be desirable to provide adhesion molecules back to the cornea during the refractive procedure. For example, an adhesion eye drop solution may be administered prior to, or immediately following ablation and prior to, or after, the resetting of the epithelial flap. In this way, natural adhesion may be restored. Classes of suitable adhesion molecules include, but are not limited to, the selecting, the integrins, and the cadherins. Examples of adhesion molecules within these classes include, but are not limited to, Collagen types I-XI, fibronectin, laminin, E-cadherin, vitronectin, and the like. Mixtures of adhesion molecules may also be desirable.

It may be that the delaminating device has damaged the basal epithelial cells, or the entire epithelial layer completely. Lubrication of the cornea during the delamination procedure may help to ameliorate this problem. For example, a lubricating substance may be added to the delaminating device, or put on the cornea directly (e. in eye drop form). Any suitable lubricant may be used. For example, the lubricant may be a viscoelastic aqueous polymer, or combination of polymers. Examples of suitable lubricants include, but are not limited to, polyacrylic acid, polyacrylimide, carboxymethylcellulose, hyaluronic acid, and the like. Mixtures of these lubricants may also be suitable.

Another treatment procedure involves cooling the eye, cornea, or epithelium, before, during, or after the delaminating procedure, for example, by cooling the temperature of the device during the delaminating procedure. Cooler temperatures limit the scope of potential injury caused to the epithelium. Alternatively, cooling fluids may be added to the eye, cornea, or epithelium, before, during, or after the delaminating procedure. Therefore, the delaminating device may be cooled in order to help reduce the amount of injury caused to the epithelium. In any event, the extent of damage to the epithelium may be minimized by avoiding excessive drying, wiping, or irrigation of the cornea during the refractive procedure.

Another treatment regime is the introduction of an IL-1 receptor agonist, or a FAS receptor agonist beneath the corneal epithelium prior to, during, or after the delaminating procedure. For example, it is thought that (IL-1) alpha and IL-1 beta are released from the corneal epithelial cells, upon injury, which may stimulate apoptosis.

Another treatment includes reinstituting the nutrient supply chain from the aqueous humor to the epithelial layer. Without a constant supply of oxygen and nutrients, epithelial cells die. One procedure involves providing an active depot of agents to supply the epithelial layer with the needed nourishment. The nourishing agents include a variety of agents useful in nourishing the epithelium. For example, the nourishing agents may be selected from vitamins, minerals, water, salt, other nutrients, and their mixtures.

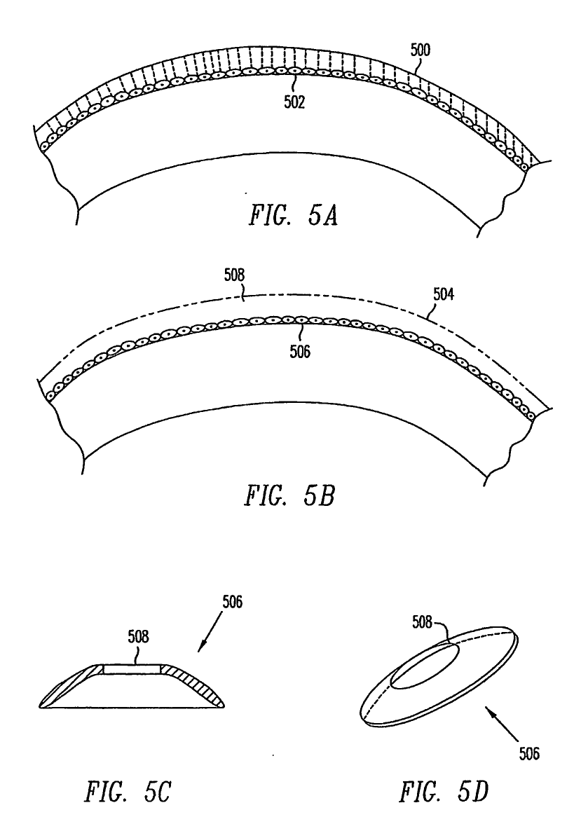

As noted above, bandage contact lenses are sometimes used to aid the healing process. To assure that such lenses allow nutrient or oxygen flow to the epithelium from the surrounding environment, one may alter the structure of the bandage contact lenses traditionally used. For example, suitable modifications to traditional bandage contact (e. those made of silicone based hydrogels and other accepted polymeric materials) lenses are shown in FIGS. 5A and 5B. My bandage contact lenses are shown in FIGS. 5C and 5D. As shown in FIG. 5A, a bandage contact lens (500) is placed on top of an epithelial layer (502). Here, the bandage contact lens (500) may not be constructed of the same material as traditional soft contact lenses, which are often employed as bandages. Instead, the bandage contact lens (500) may be constructed of a material provided with enhanced permeability to the flow of nutrients therethrough compared to traditional soft contact lens. For example, certain mesh or screen-like materials may be useful. Similarly, reticulated polymeric structures, perhaps of gels, may be used.

Another way in which the traditional bandage contact lenses may be modified is shown in FIG. 5B. Shown there is a traditional bandage contact lens (504), covering the epithelial layer (506). The bandage contact lens (504) has been modified to provide at least one hole, slit, perforation, or opening therethrough. In this way, oxygen and nutrients may pass to the epithelium.

FIG. 5C shows a cross-section of my bandage contact lens (506) having opening (508) often in the general center of the lens. The back side of the lens is generally substantially a shape conform to the eye surface. The bandage contact lens and may be treated with a number of materials, e. g., agents. In addition, the lens may be infused or treated with other suitable or desirable materials including antibiotics and nutrient materials as are discussed elsewhere in this disclosure. FIG. 5D shows, for completeness, a perspective view of the contact lens shown in FIG. 5C. These bandage lenses have special use when used in conjunction with a procedure in which an epithelial flap is lifted and a laser-based procedure practiced in the denuded area before the epithelial flap is replaced. In such a procedure, my bandage lens is placed over the replaced epithelium tissue. In general, the outer diameter of the lens should be sufficient to cover the edges of the lifted-and-replaced epithelium and so prevent the conjunctiva of the eyelid from contacting that edge. In turn, the diameter of the opening should be sufficient to allow that conjunctiva to contact the epithelium over the region of the cornea where the laser procedure has taken place. For instance, many laser corneal reformation devices utilize a corneal region having a diameter of 5 to 7 mm. Consequently, a hole or opening of about 9 mm diameter is suitable to allow contact of the epithelium over the laser treated area with the eyelid's conjunctiva. a suitable range of opening would be 6-10 mm. The opening (508) in the lens (506) may have an area that is variously more than 10%, more than 20%, more than 25%, more than 30%, and may be more than 40% of the area of the front of the lens were the lens not to have the hole.

Suitable polymers for the lens include various hydrophilic polymers such as hydroxyethylmethacrylate, polyvinyl alcohol, lidofilcon, polyethyleneoxide, poly n-vinyl pyrrolidone, gelatin, collagen, polymerized hyaluronic acid (cross-linked or not), and chondroitan sulfate. Often, I have found it desirable to increase the physical porosity of the polymer to increase its functionality as a bandage lens. Formation of the lens using two-phase interpenetrating networks, ablation with lasers or the like to produce pinholes for added porosity, and molding the lens with small mandrels to produce pinhole porosity are all procedures suitable for producing the added porosity.

In the event that the delamination or ablation procedures have interfered with the signal transduction pathways among or between the various corneal cells, and therefore caused epithelial flap cell death, application of suitable pharmacological agents is desired. That is, these procedures could have altered a single molecule within the cornea, which in turn had the domino effect of producing epithelial cell apoptosis. Correction of improper signaling between the cells may be accomplished by the administration of a pharmacological agent that produces proper signaling.

Still another treatment procedure includes the introduction or growth factors during, after, or prior to the delaminating procedure. Hepatocyte growth factor and keratinocyte growth factor are paracrine growth factors produced by fibroblast cells, which modulate epithelial cells. These growth factors are secreted by keratocytes and they regulate wound healing and homeostatic functions in the epithelial cells. For example, and keratinocyte growth factor may stimulate cell proliferation. Similarly, hepatocyte growth factor may inhibit corneal epithelial cell differentiation. Therefore, one treatment regime includes the concurrent stimulation of epithelial cell proliferation, with the inhibition of epithelial cell differentiation.

Epithelial wound healing over a non-epithelialized surface is dependent on the function of the epithelial cell. So-called “healing” epithelial cells are functionally and phenotypically different from epithelial cells in homeostasis (normally residing in an undamaged epithelium). Epithelial cells in homeostatis proliferate at the basal cell layer, at a low rate and terminally differentiate as daughter cells are pushed inward, and upward, towards the epithelial surface. At the basal cell layer, one major function is the production of more epithelial cells. This is non-proteolytic, non-remodeling, and simply provides for a maintenance state.

Healing epithelial cells, on the other hand, are phenotypically and functionally different from homeostatic epithelial cells. Healing epithelial cells are undergoing migration and remodeling of the substrate onto which they are moving. Healing epithelial cells dissolve their intercellular attachments (desmosomes) and produce actin filaments for locomotive capability. In addition to migration, healing epithelial cells are resorbing/dissolving nonviable substratum from viable substratum. As such, these cells are producing proteases (e.g., intersital collagenase, plasminogen activator, and matrix metalloproteinases).

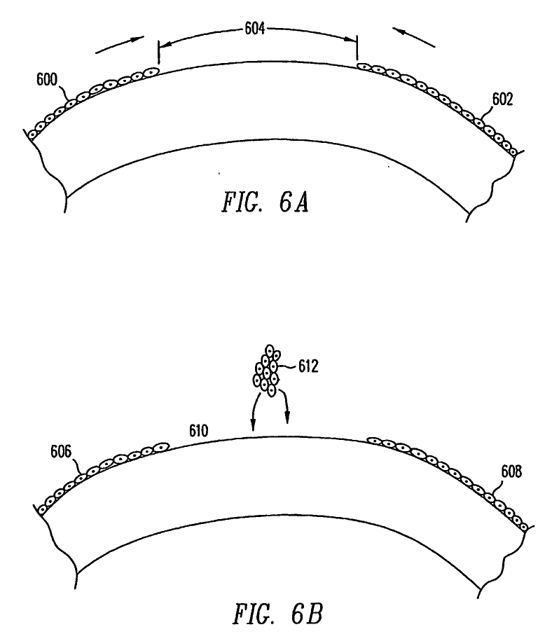

Another treatment method makes use of the differences in the homeostatic epithelial cells and the healing epithelial cells. Illustrative uses are depicted in FIGS. 6A and 6B. Shown in FIG. 6A, the peripheral epithelial cells (600, 602) are intact, and may likely be in a homeostatic state. At least a portion of the intact epithelial cells may be moved to cover the ablated area (604) to aid in the healing process. Similarly, as shown in FIG. 6B, at least a portion of the intact homeostatic epithelial cells (606, 608) may be removed and introduced (612) onto the ablated stromal area (610) perhaps in a concentrated fashion.

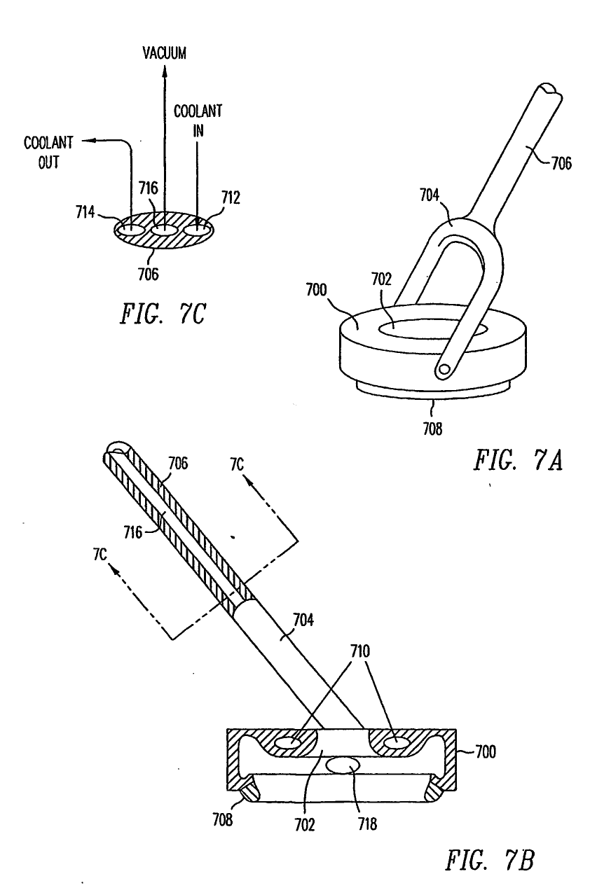

FIG. 7A shows a perspective view of a vacuum device or suction ring having an integrated coolant flow that is suitable for use in a delaminating procedure. FIG. 7B shows a cross-section of the FIG. 7A device and FIG. 7C shows a cross-section of the handle of the device showing positioning of coolant flow passageways and vacuum access lines.

FIG. 7A shows a suction ring (700) having an opening (702) through which a cornea may be seen during an epithelial procedure. Also shown is a yoke (704) supported by a handle (706) through which coolant and vacuum are accessed. Vacuum rings such as that seen in these figures are, in many ways, similar to others known in this area of surgery. This one may be of a size and type allowing the cornea on the operative eye to be flattened it may be of the size and type that creates sizable extension of the front of the eye after the vacuum ring (700) is placed on the eye. In this particular instance, the ring itself (700) acts as a closed heat exchanger for a flowing coolant, in addition to providing a stable surface upon which to carry out the epithelial delamination procedure.

It is to be noted that FIGS. 7A and 7B show the presence of a rubber skirt (708) situated on the lower side of the vacuum ring (700) that renders the fit and comfort of the vacuum ring (700) to be more appreciated.

Shown in FIG. 7B is a cross-section of vacuum ring (700) showing a fluid passageway (710) that generally encircles the opening (702) in the top center of ring (700). In this variation, coolant flows through handle (706) into arm of yoke (704) and into fluid passageways (710) and returns through the other side of the yoke and is returned through the handle (706). FIG. 7C shows passageway (712) in handle (706) and return coolant passageway (714). FIGS. 7B and 7C further show vacuum access passageway (716) that opens into the eye-side portion of vacuum ring (700) through opening (718).

The coolant fluid passageway (710) around the vacuum ring opening (702) forms an indirect heat exchanger and permits cooling, even chilling, of the region of the eye where the epithelial layer is pushed about. In the case of an epithelial flap, the coolant is very near where the epithelium is maintained before, during, and occasionally after the movement of the epithelium from the corneal surface. This provides an amount of cooling material in close proximity to the site where that epithelial tissue is maintained prior to its replacement on the eye.

As noted above, this chilled fluid or coolant may be maintained in the range of just above 0° C. up to about 10° C. In some instances, chilled fluid up to 18° C. or 20° C. may be used although we have found best effects at a neighborhood of 10° C.

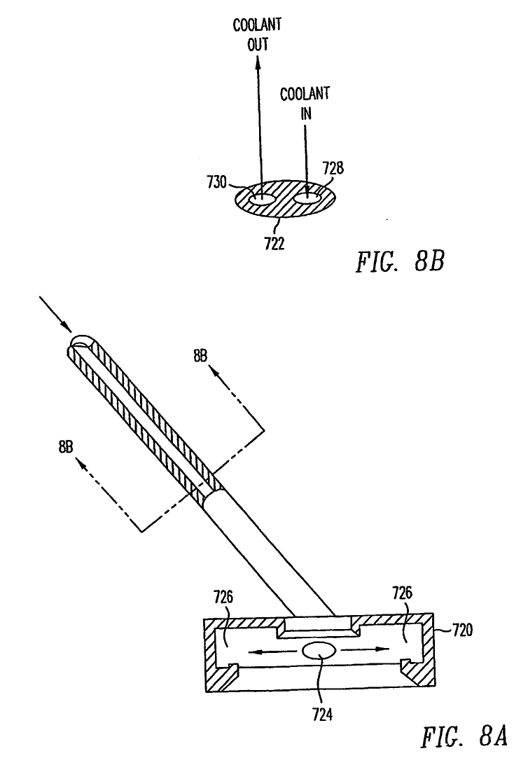

FIGS. 8A and 8B show a variation of the cooling device in which a chilling fluid is used as the vacuum source. FIG. 8A shows a cross-section of the vacuum device through vacuum ring (720) and FIG. 8B shows a cross-section of the supply handle (722) with integral coolant passageways.

FIG. 8A shows vacuum ring (720) having an opening (724) through which coolant enters vacuum ring (720). The fluid is allowed to migrate around the open area (726) around the periphery of the cornea on the scleral surface. By proper control of liquid flows in and out of vacuum ring (720), the ring is maintained in a slight vacuum thereby pulling the vacuum ring (720) against the surface of the eye. Such a vacuum system may be started on gas such as air or nitrogen and then changed over to a cooled liquid (such as a saline solution) during the course of the procedure. This is an open coolant flow and indeed may be of a mixed phased nature, maintaining air at least partially in such a system. FIG. 8B shows the inward coolant flow passageway (728) and the exiting fluid passageway (730). As will be appreciated, it is unlikely that the fluid flow will be very high since the heat load on a person's eyeball is not very high.

FIG. 9 shows a partial cross-sectional view of a vacuum ring or aplanation device (736) having a vacuum ring (738) with a plenum region (740) and a vacuum access port (742) allowing the vacuum to interact with the eye and hold the ring firmly to the front surface of an eye. The central hole (744) that is positioned over the cornea during the de-epithelialization or epithelium lifting procedure is seen as is the handle (746) having integral vacuum line (748) passing therethrough.

This variation involves the presence of a spray nozzle (750) that allows a mist or fine spray to pass onto the surface of the eye and particularly onto the epithelium pre- or post-separation. Fluid line (752) is shown passing the handle as well.

Each of the variations of the vacuum ring and aplanators shown herein also include as a component, a vacuum source independently, a chilled fluid source.

The variation shown in FIG. 9 involves the introduction of a non-confined fluid onto the surface of the eye. It may be the case that a dam in the form of a ring or the like such as is shown in FIG. 10 and discussed below may be useful for maintaining the free coolant fluid in the region of the epithelium. Again, this may be desirable, but not necessary, if the procedure involved is sufficiently short or the presence of the fluid is not objectionable during the procedure.

FIG. 10 shows a cross-section of a cooling device (754) having variously the typical vacuum ring functions and the ability to provide coolant liquid to the surface of the eye before, during, and after the procedure involved.

The vacuum ring (756) with the vacuum port (758) leading to a vacuum source through the handle or other support (760) is shown and has been discussed before. In this variation a coolant fluid line having a distal opening (762) opening above the opening (764) above the cornea is shown. In this variation (754), an amount of coolant fluid passes through coolant line (760) and out through coolant port (762) onto the comea and epithelium during the relevant procedure. A ring or dam (765) that may be either permanently affixed to vacuum ring (756) or temporarily affixed as needed or desired by the user. The coolant fluid provided through opening (762) may be continuous or on an as-needed basis often to be controlled by the user.

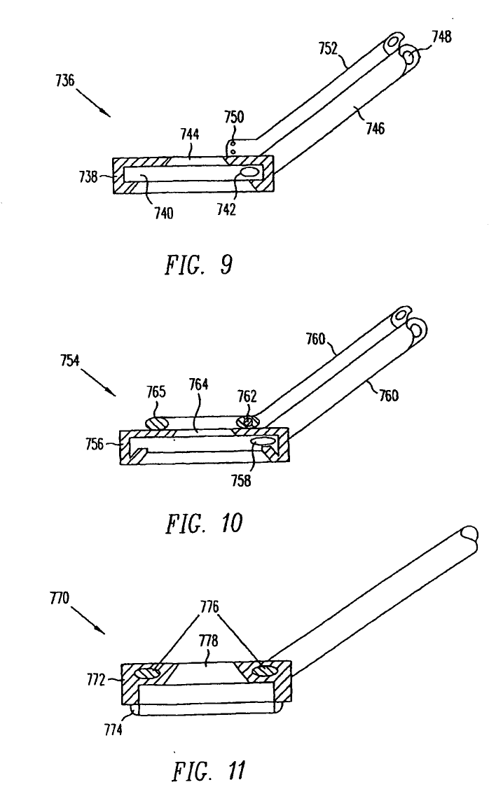

FIG. 11 shows a further variation of cooling device (770) in which vacuum ring (772), shown in side cross-section with a polymeric skirt (774), includes a captured supply of a heat storage material (776). In this variation, the heat storage material (776) is situated in a ring about the corneal opening (778) and serves to provide cooling to the surface by direct heat exchange. A desirable material for the heat storage material (776) is a material that undergoes a phase change in the region below the temperature of the human body. Various eutectic salt mixtures are known and may be specifically tailored for the service these devices would be cooled prior to use to change the heat storage material to a proper chilled salt phase and, during the procedure, the salt mixture would change crystal phase or undergo a solid-to-liquid phase change.

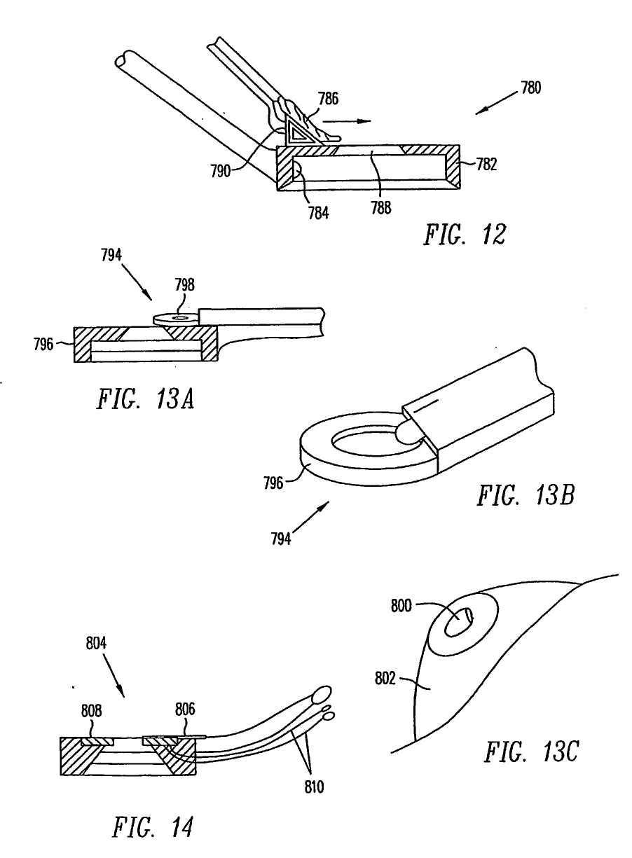

FIG. 12 shows in schematic cross-section, a device for delaminating the epithelial layer from the eye. This combination device (780) includes a vacuum ring (782) with vacuum port (784), each of which has been discussed above at length. Again, this vacuum ring may be of the type that provides aplanation or provides the ability to raise the surface of the cornea for a different approach angle to the epithelium. Depending on a variety of corneal parameters, the approach of a blunt dissector may be at a high angle or at a low angle depending upon the procedure involved. Again, separation of the epithelial layer without the inclusion of any of the corneal tissue below is the goal of such a device. In this variation, the blunt dissector blade is shown to move across the opening (788} in vacuum ring. Adjacent the dissector blade (786) may be seen a indirect heat exchange member (790) having coolant flow through it and thereby cooling the dissector blade (786).

FIGS. 13A and 13B show a variation of a delaminator assembly (794) in which vacuum ring (796) is coupled with a blunt dissector blade (798) that oscillates to produce a pocket (800) beneath the epithelial layer of an eye as shown in FIG. 13C. The coolant fluid in such a variation may be introduced into or onto or above the eye using any of the exchanger variations shown just above. The delaminator or blunt dissector blade (798) may also be used to distribute any of the materials discussed elsewhere, e.g., effective treatment agents such as adhesion molecules, lubricants, nourishing agents, IL-1 receptor agonist, growth factors, and mixtures in combination with sources of those agents.

FIG. 14 shows a variation of my device (804) having a vacuum ring (806) and using a thermoelectric cooling device, e. g., a Peltier device, powered by wires (810) to provide cooling to the vicinity of the eye where needed.

Although illustrative variations of the described methods have been set forth in detail above, it will be evident to one skilled in the art that various changes and modifications may be made without departing from the spirit of the invention, the scope of which, is set forth in the following claims.

1-12. (canceled)

13. A device for treating an eye comprising
an epithelial delaminator configured for separating an epithelial flap from the eye and for cooling the eye, epithelium, or cornea, before, during, or after the delamination.

14. The device of claim 13 wherein the epithelial delaminator configured for cooling the eye, epithelium, or cornea, before, during, or after the delamination is configured for cooling the epithelial delaminator to an effective temperature during the delamination.

15. The device of claim 13 wherein the epithelial delaminator configured for cooling the eye, epithelium, or cornea, before, during, or after the delamination is configured for introducing a cool fluid to the eye, epithelium, or cornea, before, during, or after the delamination.

16. A method for treating an eye, comprising the steps separating an epithelial flap from the eye, and applying a nutrient permeable, bandage contact lens to a cornea having a delaminated and replaced epithelium.

17. The method of claim 16 wherein the bandage contact lens has at least one opening therethrough.

18. The method of claim 16 wherein the bandage contact lens is comprised of a mesh or screen-like material.

19-47. (canceled)

48. A bandage contact lens comprising a polymeric, substantially round lens having a convex front surface, a concave rear surface, the concave rear surface being configured to contact and to substantially match the front surface of an eye, and having at least one opening with a continuous edge between the front surface and the rear surface.

49. The contact lens of claim 48 wherein the opening is substantially round.

50. The contact lens of claim 48 wherein the opening is substantially centered in the lens.

51. The contact lens of claim 49 wherein the opening is substantially centered in the lens.

52. The contact lens of claim 48 wherein the area of the opening is more than 10% of the area of the front surface of the lens were the lens not to have the opening.

53. The contact lens of claim 48 wherein the area of the opening is more than 20% of the area of the front surface of the lens were the lens not to have the opening.

54. The contact lens of claim 48 wherein the area of the opening is more than 25% of the area of the front surface of the lens were the lens not to have the opening.

55. The contact lens of claim 48 wherein the area of the opening is more than 30% of the area of the front surface of the lens were the lens not to have the opening.

56. The contact lens of claim 48 wherein the lens has substantial physical porosity.

57. The contact lens of claim 48 wherein the lens comprises a mesh or screenlike material.

58. The contact lens of claim 48 wherein the lens comprises a reticulated polymeric structures.
